(19) United States
(12) Patent Application Publication
(10) Pub. No.: US 2025/0120618 A1
(43) Pub. Date: Apr. 17, 2025

(54) NON-INVASIVE BLOOD GLUCOSE MONITORING MODULE WITH HIGH OPTICAL TRANSMITTANCE

(71) Applicant: Taiwan-Asia Semiconductor Corporation, Hsinchu City (TW)

(72) Inventors: Hsiu-Jung Huang, Hsinchu City (TW); Sheng-Wei Chen, Hsinchu City (TW)

(21) Appl. No.: 18/768,485

(22) Filed: Jul. 10, 2024

(30) Foreign Application Priority Data
Oct. 14, 2023 (TW) 112139278

(51) Int. Cl. A61B 5/145; A61B 5/1455; H01L 25/16

(52) U.S. Cl. CPC A61B 5/14532; A61B 5/1455; A61B 2562/185; H01L 25/167

(57) ABSTRACT

A non-invasive blood glucose monitoring module with high optical transmittance includes a substrate, a first detecting unit and a second detecting unit. The first detecting unit includes a first light source set, a first photosensitive element and first blocking wall structures. The first photosensitive element receives light, with a first wavelength, emitted from the first light source set. The first blocking wall structures are located between the two first light-emitting elements and between the first light source set and the first photosensitive element. The second detecting unit includes a second light source set, a second photosensitive element and second blocking wall structures. The second photosensitive element receives light, with a second wavelength, emitted from the second light source set. The second blocking wall structures are located between the two second light-emitting elements and between the second light source set and the second photosensitive element.

### CROSS REFERENCE TO RELATED APPLICATION

This application claims the benefit of Taiwan Patent Application Serial No. 112139278 filed on Oct. 14, 2023. The entirety of each Application is incorporated herein by reference.

### BACKGROUND OF THE INVENTION

### 1. Field of the Invention

The present invention relates to a non-invasive blood glucose monitoring device, especially a non-invasive blood glucose monitoring module with high optical transmittance and with improved detection accuracy.

### 2. Description of Related Art

In conventional manufacturing steps of a non-invasive blood glucose monitoring device, a light-emitting element and a photosensitive element on a substrate are first packaged through a packaging material, and then a cover is disposed on the packaging material. Next, grooves are formed at appropriate locations of the cover and the packaging material (such as between the light-emitting element and the photosensitive element) by half-cutting, and at last the grooves are filled with an opaque glue material by dispensing, so as to form necessary blocking walls after the opaque glue material being solidified.

However, in proceeding the half-cutting process, it is difficult to control cutting depths and paths of the cutting tools, which may cause damages to the cover or the substrate. Furthermore, since the blocking walls with heights reaching the surface of the cover, a path of light from the light-emitting element to entering the light-emitting element is thus reduced, which influences the light-emitting or light-receiving effects. In addition, conventional packaging materials are mostly epoxy resins, generally with optical transmittance of less than 80%, which also affects the light-emitting or light-receiving effects, and thereby affecting the accuracy of blood glucose detection.

In light of this, it is really worthy of research and development, for solving those above-mentioned problems, to design a non-invasive blood glucose monitoring module.

### SUMMARY OF THE INVENTION

An objective of the present invention is to provide a non-invasive blood glucose monitoring module with high optical transmittance and with improved detection accuracy.

To achieve the above mentioned objectives, the non-invasive blood glucose monitoring module with high optical transmittance according to the present invention comprises a substrate, a first detecting unit and a second detecting unit. The first detecting unit includes a first light source set, a first photosensitive element and a plurality of first blocking wall structures. The first detecting unit has two first light-emitting elements and each first light-emitting element emits light with a first wavelength. The first photosensitive element correspondingly receives the light emitted from the first light source set, and each of the first light-emitting elements has a different minimum distance relative to the first photosensitive element. The plurality of first blocking wall structures are located between the two first light-emitting elements and between the first light source set and the first photosensitive element. The second detecting unit includes a second light source set, a second photosensitive element and a plurality of second blocking wall structures. The second light source set has two second light-emitting elements, each second light-emitting element emits light with a second wavelength, and the second wavelength is different from the first wavelength. The second photosensitive element correspondingly receives the light emitted from the second light source set, and each of the second light-emitting elements has a different minimum distance relative to the second photosensitive element. The plurality of second blocking wall structures are located between the two second light-emitting elements and between the second light source set and the second photosensitive element.

In one embodiment of the present invention, a length of each of the first blocking wall structures is longer than a length of each of the first light-emitting elements and longer than a length of the first photosensitive element, and a length of each of the second blocking wall structures is longer than a length of each of the second light-emitting elements and longer than a length of the second photosensitive element.

In one embodiment of the present invention, any first blocking wall structure of the first detecting unit is not connected to the correspondingly closest one of the second blocking wall structures of the second detecting unit, thus to maintain a gap thereof.

In one embodiment of the present invention, the first photosensitive element and the first light-emitting elements of the first detecting unit are arranged along a first straight line, the second photosensitive element and the second light-emitting elements of the second detecting unit are arranged along a second straight line, and the first straight line is parallel to the second straight line.

In one embodiment of the present invention, the first detecting unit further includes a third light source set, the first light source set and the third light source set are arranged symmetrically relative to the first photosensitive element, the third light source set has two third light-emitting elements, and each of the third light-emitting elements emits light with the first wavelength or the second wavelength. And the second detecting unit further includes a fourth light source set, the second light source set and the fourth light source set are arranged symmetrically relative to the second photosensitive element, the fourth light source set has two fourth light-emitting elements, each of the fourth light-emitting elements emits light with the first wavelength or the second wavelength, and the wavelength of the light emitted from each of the fourth light-emitting elements is different from the wavelength of the light emitted from each of the third light-emitting elements.

In one embodiment of the present invention, a viscosity of the material of the first blocking wall structures and the second blocking wall structures is greater than 10000 cps.

In one embodiment of the present invention, the non-invasive blood glucose monitoring module with high optical transmittance further comprises a packaging structure to package each of the first light-emitting elements, the first photosensitive element, each of the second light-emitting elements and the second light-emitting element. And a height of the package structure is larger than heights of each of the first light-emitting elements, of the first photosensitive element, of each of the second light-emitting elements and of the second light-emitting element.

In one embodiment of the present invention, the packaging structure, each of the first blocking wall structures and each of the second blocking wall structures have the same height.

In one embodiment of the present invention, a packaging material of the packaging structure has a viscosity between 1000 cps and 3000 cps and has an optical transmittance greater than 95%.

In one embodiment of the present invention, the first wavelength is between 1000 nm and 1400 nm and the second wavelength is between 1400 nm and 1800 nm.

In one embodiment of the present invention, the non-invasive blood glucose monitoring module with high optical transmittance further comprises a transparent cover disposed on the first detecting unit and the second detecting unit.

### BRIEF DESCRIPTION OF THE DRAWINGS

FIG. 1 is a top view of a first embodiment of a non-invasive blood glucose monitoring module with high optical transmittance according to the present invention;

FIG. 2A is a cross-sectional view along a first straight line L1 shown in FIG. 1 of the first embodiment of the non-invasive blood glucose monitoring module with high optical transmittance according to the present invention;

FIG. 2B is a cross-sectional view along a second straight line L2 shown in FIG. 1 of the first embodiment of the non-invasive blood glucose monitoring module with high optical transmittance according to the present invention;

FIG. 3 is a top view of a second embodiment of the non-invasive blood glucose monitoring module with high optical transmittance according to the present invention;

FIG. 4A is a cross-sectional view along the first straight line L1 shown in FIG. 3 of the second embodiment of the non-invasive blood glucose monitoring module with high optical transmittance according to the present invention;

FIG. 4B is a cross-sectional view along the second straight line L2 shown in FIG. 3 of the second embodiment of the non-invasive blood glucose monitoring module with high optical transmittance according to the present invention.

### DETAILED DESCRIPTION OF THE PREFERRED EMBODIMENT

Since various examples and embodiments in the present invention are only illustrative and non-restrictive, a person skilled in the art can easily conceive other examples and embodiments without contravening the scope of the present invention, after reading this specification, and can make the features and advantages of these embodiments more evident based on the following detailed description and claims.

Herein, the description of unit, element and component in the present invention uses “one”, “a”, or “an”. This is for convenience and for offering general meaning of the category of the present invention. Therefore, the description should be understood as including “one”, “at least one”, and singular and plural forms at the same time unless the context clearly indicates otherwise.

Herein, the description of the terms “first” or “second” and similar ordinal numbers are mainly used to distinguish or refer to the same or similar elements or structures and do not necessarily imply that such components or structures are spatially or temporally distinct order. It should be understood that ordinal numbers, in certain situations or configurations, may be used interchangeably without affecting the implementation of the present invention.

Herein, the description of “comprise”, “have” or other similar semantics have the non-exclusive meaning. For example, components or structures with a plurality of elements are not only limited to those disclosed in this specification, but also include generally inherent elements, which are not explicitly listed here for the components or the structures.

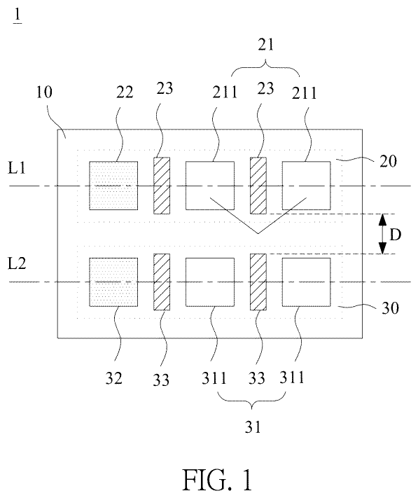

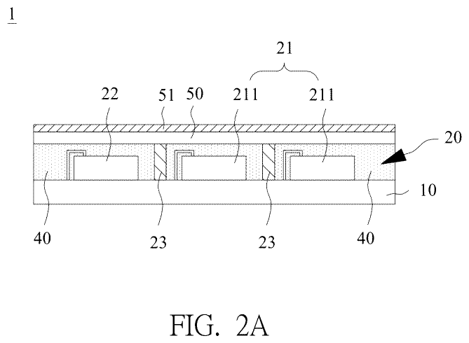

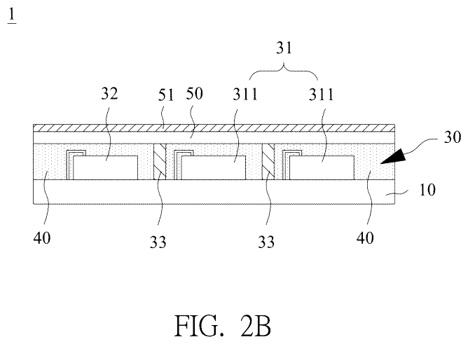

Please refer to FIG. 1, FIG. 2A and FIG. 2B together, wherein FIG. 1 is a top view of a first embodiment of a non-invasive blood glucose monitoring module with high optical transmittance according to the present invention, FIG. 2A is a cross-sectional view along a first straight line L1 shown in FIG. 1 of the first embodiment of the non-invasive blood glucose monitoring module with high optical transmittance according to the present invention, and FIG. 2B is a cross-sectional view along a second straight line L2 shown in FIG. 1 of the first embodiment of the non-invasive blood glucose monitoring module with high optical transmittance according to the present invention. As shown in FIG. 1, FIG. 2A and FIG. 2B, in this embodiment, the non-invasive blood glucose monitoring module with high optical transmittance 1 according to the present invention comprises a substrate 10, a first detecting unit 20 and a second detecting unit 30, and the first detecting unit 20 and the second detecting unit 30 are disposed on the substrate 10. The substrate 10 is a basic structure component of the non-invasive blood glucose monitoring module with high optical transmittance 1, for carrying other components of the non-invasive blood glucose monitoring module with high optical transmittance 1. In the present invention, the substrate 10 is a ceramic substrate, a silicon substrate or a PCB substrate, but not limited thereto. The substrate 10 can also be made of other materials.

The first detecting unit 20 includes a first light source set 21, a first photosensitive element 22 and a plurality of first blocking wall structures 23. The first light source set 21 mainly emits light to skins, and the first light source set 21 has two first light-emitting elements 211. A default distance is set between the two first light-emitting elements 211, and each first light-emitting element 211 emits light with a first wavelength. In the present invention, a single LED is adopted as a light source of each of the first light-emitting elements 211, but the type of the first light-emitting element 211 can be changed according to different design requirements. The first light-emitting element 211 can be electrically connected to the substrate 10 through a bonding wire. In this embodiment, the first wavelength is between 1000 nm and 1400 nm, and preferably is 1065 nm, but not limited thereto.

The first photosensitive element 22 mainly receives light which is diffusely reflected by the skins and then transmitted back. The first photosensitive element 22 is located on one side of the first light source set 21 to correspondingly sense the light with the first wavelength emitted by the first light source set 21. In the present invention, a single photo detector is adopted for the first photosensitive element 22, but the type of the first photosensitive element 22 can be changed according to different design requirements. The first photosensitive element 22 can also be electrically connected to the substrate 10 through a bonding wire. Each first light-emitting element 211 has a different minimum distance relative to the first photosensitive element 22 (that is, a linear distance from a center of the first light-emitting element 211 to a center of the first photosensitive element 22). That is to say, one of the two first light-emitting elements 211 is closer to the first photosensitive element 22 than the other first light-emitting element 211. In this embodiment, the first photosensitive element 22 and the first light-emitting elements 211 of the first detecting unit 20 are arranged along the first straight line L1.

The plurality of first blocking wall structures 23 mainly provide an effect of light-blocking, and the plurality of first blocking wall structures 23 are located between the two first light-emitting elements 211 of the first light source set 21 and between the first light source set 21 and the first photosensitive element 22. In design, the first light source set 21 and the first photosensitive element 22 are separated by the first blocking wall structure 23 to prevent the first photosensitive element 22 from directly receiving light emitted by the first light source set 21 but not reflected by the skins, thereby improving the accuracy of detecting results. Through separating the two first light-emitting elements 211 by the first blocking wall structure 23, interference in luminous intensity between the two first light-emitting elements 211 can also be avoided. The plurality of first blocking wall structures 23 can be made of non-transparent (including visible light and invisible light, and with an optical transmittance of 0%) materials, such as black rubber, but the invention is not limited thereto.

In the present invention, a viscosity of the material of the plurality of first blocking wall structures 23 is greater than 10000 cps. With this design, when the material for forming the first blocking wall structures 23 is applied to a surface of the substrate 10 by dispensing, a three-dimensional structure can be directly formed on the substrate 10, due to the high viscosity of the material, without further processes such as cutting by cutting tools and potting, which makes the manufacturing steps of the plurality of first blocking wall structures 23 more simplified. A length of each of the first blocking wall structures 23 is slightly longer than a length of each of the first light-emitting elements 211 and longer than a length of the first photosensitive element 22. That is to say, the length of each first blocking wall structure 23 will be adjusted according to different sizes of the first light-emitting element 211 and the first photosensitive element 22 (for example, the length of each first blocking wall structure 23 is approximately the length of the first light-emitting element 211 plus about 0.2 mm or the length of the first photosensitive element 22 plus about 0.2 mm). A width of each blocking wall structure 23 is about 0.1 mm to 0.3 mm, and a height of each first blocking wall structure 23 is higher than a height of the first light-emitting element 211 and the corresponding bonding wire, or higher than a height of the first photosensitive element 22 and the corresponding bonding wire (for example, each height of the first blocking wall structure 23 is approximately 0.3 mm).

The second detecting unit 30 includes a second light source set 31, a second photosensitive element 32 and a plurality of second blocking wall structures 33. The second light source set 31 mainly emits light to the skins, and the second light source set 31 has two second light-emitting elements 311. A default distance is set between the two second light-emitting elements 311, and each second light-emitting element 311 emits light with a second wavelength. The second wavelength is different from the aforementioned first wavelength. In the present invention, a single LED is adopted as a light source of each of the second light-emitting elements 311, but the type of the second light-emitting element 311 can be changed according to different design requirements. The second light-emitting elements 311 can be electrically connected to the substrate 10 through bonding wires. In this embodiment, the second wavelength is between 1400 nm and 1800 nm, and preferably is 1650 nm, but not limited thereto. In addition, depending on different designs, the value of the first wavelength and the value of the second wavelength can also be interchanged with each other.

The second photosensitive element 32 mainly receives light which is diffusely reflected by the skins and then transmitted back. The second photosensitive element 32 is located on one side of the second light source set 31 to correspondingly sense the light with the second wavelength emitted by the second light source set 31. In the present invention, a single photo sensor is adopted for the second photosensitive element 32, but the type of the second photosensitive element 32 can be changed according to different design requirements. The second photosensitive element 32 can also be electrically connected to the substrate 10 through a bonding wire. A minimum distance between each second light-emitting element 311 and the second photosensitive element 32 (that is, a linear distance from a center of the second light-emitting element 311 to a center of the second photosensitive element 32) is different. That is to say, one of the two second light-emitting elements 311 is closer to the second photosensitive element 32 than the other second light-emitting element 311. In the present invention, the second photosensitive element 32 and each of the second light-emitting elements 311 of the second detecting unit 30 are arranged along the second straight line L2, and the second straight line L2 is parallel to the first straight line L1. Accordingly, the first detecting unit 20 and the second detecting unit 30 are arranged side by side with each other. That is to say, the two first light-emitting elements 211 of the first light source set 21 and the first photosensitive element 22 of the first detecting unit 20, and the two second light-emitting elements 311 of the second light source set 31 and the second photosensitive element 32 of the second detecting unit 30 can form an arrangement structure similar to a 2×3 matrix. The first detecting unit 20 and the second detecting unit 30 each forms an x-direction row, while the first photosensitive element 22 and the second photosensitive element 32, each first light-emitting element 211 and the adjacent second light-emitting element 311 respectively form a y-direction row.

The plurality of second blocking wall structures 33 mainly provide an effect of light-blocking, and the plurality of second blocking wall structures 33 are located between the two second light-emitting elements 311 of the second light source set 31 and between the second light source set 31 and the second photosensitive element 32. In design, the second light source set 31 and the second photosensitive element 32 are separated by the second blocking wall structure 33 to prevent the second photosensitive element 32 from directly receiving light emitted by the second light source set 31 but not reflected by the skins, thereby improving the accuracy of detecting results. Through separating the two second light-emitting elements 311 by the second blocking wall structure 33, interference in luminous intensity between the two second light-emitting elements 311 can also be avoided. The plurality of second blocking wall structures 33 can be made of non-transparent (including visible light and invisible light, and with an optical transmittance of 0%) materials, such as black rubber, but the invention is not limited thereto.

In the present invention, the material properties of the plurality of second blocking wall structures 33 are the same as the material properties of the aforementioned plurality of first blocking wall structures 23, and a designed size of each second blocking wall structure 33 will also be adjusted as sizes of the light-emitting element 311 and the second photosensitive element 32 changed. That is similarly to the above-mentioned first blocking wall structure 23 and no further details will be given here. In the present invention, it is worth mentioning that any first blocking wall structure 23 of the first detecting unit 20 is not connected to the correspondingly closest one of the second blocking wall structures 33 of the second detecting unit 30, thus to maintain a gap D thereof (as shown in FIG. 1, the gap D is formed between any first blocking wall structure 23 and the second blocking wall structure 33 located on the same y-direction row). Through setting the aforementioned gap D, a trace spacing for the bonding wire is created between the first blocking wall structure 23 and its corresponding second blocking wall structure 33, so that the circuit configuration of the monitoring module can be flexibly adjusted.

The non-invasive blood glucose monitoring module with high optical transmittance 1 of the present invention further comprises a packaging structure 40. The packaging structure 40 is used to package all electrical components disposed on the substrate 10, including two first light-emitting elements 211 of the first light source set 21 and the first photosensitive element 22, two second light-emitting elements 311 of the second light source set 31 and the second photosensitive element 32, and corresponding bonding wires, etc. For the packaging structure 40, a packaging material is applied to the surface of the substrate 10, so that any or a combination of the first light-emitting elements 211, the first photosensitive element 22, the second light-emitting elements 311, the second photosensitive element 32 and the corresponding bonding wires will be completely embedded in the packaging material. Then, after compression molding and solidifying of the packaging material, the packaging structure 40 forms. Since the bonding wires are connected to the substrate 10 from a surface of any of the aforementioned light-emitting elements or photosensitive elements, in design, a height of the package structure 40 will be larger than those heights of each of the first light-emitting elements 211, of the first photosensitive element 22, of each of the second light-emitting elements 311 and of the second photosensitive element 32, and will be larger enough to facilitate disposing of bonding wires. In addition, the packaging structure 40, each first blocking wall structure 23 and each second blocking wall structure 33 have the same height, but not limited thereto. In the present invention, the packaging material used in the packaging structure 40 may be transparent liquid optical glue (for example, mainly made of silicon or acrylic acid material), but not limited thereto. The viscosity of the aforementioned packaging material is between 1000 cps and 3000 cps, and the optical transmittance of the packaging material is greater than 95%.

The non-invasive blood glucose monitoring module with high optical transmittance 1 of the present invention further comprises a transparent cover 50. The transparent cover 50 is disposed on the first detecting unit 20 and the second detecting unit 30. The transparent cover 50 mainly provides a protection effect for the internal components of the monitoring module. The transparent cover 50 can be fixed by gluing. Since the packaging material forming the packaging structure 40 itself has an adhesive force, an adhesively fixing effect on the transparent cover 50 occurs by the packaging material when the transparent cover 60 is disposed. In addition, by adopting a design of the same height of the aforementioned packaging structure 40, the plurality of first blocking wall structures 23 and the plurality of second blocking wall structures 33, the transparent cover 50 can be smoothly arranged on the first detecting unit 20 and the second detecting unit 30, and there is no need to perform any cutting operation relative to the transparent cover 50. The transparent cover 50 is made of transparent materials such as glass, but the invention is not limited thereto. The transparent cover 50 further includes a coating 51, and the coating 51 is formed on a side of the transparent cover 50 facing away from the substrate 10. Through the arrangement of the coating 51, the transparent cover 50 can provide an effect for allowing light in a specific wavelength band to pass through, and can effectively block stray light in other unnecessary wavelength bands. Since the coating 51 is a structural design often used for conventional transparent covers, no further details will be given here.

When the non-invasive blood glucose monitoring module with high optical transmittance 1 of the present invention is practically operated, the first light source set 21 of the first detecting unit 20 and the second light source set 31 of the second detecting unit 30 will be sequentially driven to execute a light-emitting process, and the first photosensitive element 22 and the second photosensitive element 32 will receive the light reflected from the skins to perform blood glucose detecting. Since each first light-emitting element 211 of the first light source set 21 has the different distance relative to the first photosensitive element 22, and each second light-emitting element 311 of the second light source set 31 has the different distance relative to the second photosensitive element 32, the photosensitive element 22 and the second photosensitive element 32 can perform result analyses based on different first light-emitting elements 211 and second light-emitting elements 311 respectively, thereby obtaining an effect for blood glucose detecting with improved accuracy. In addition, due to the different wavelengths of the light emitted form the first light source set 21 of the first detecting unit 20 and from the second light source set 31 of the second detecting unit 30, the non-invasive blood glucose monitoring module with high optical transmittance 1 of the present invention can conduct blood glucose detecting based on the light of different wavelengths, thereby obtaining an effect for blood glucose detecting with improved accuracy.

Furthermore, the non-invasive blood glucose monitoring module with high optical transmittance 1 of the present invention forms a plurality of first blocking wall structures 23 and a plurality of second blocking wall structures 33 with set structural shapes and sizes on the substrate 10 by using the material with the high viscosity in advance, and then a packaging process is proceeded relative to all light-emitting elements and photosensitive elements, by adopting the high transmittance packaging material, to form the packaging structure 40, so that the packaging structure 40, the plurality of first blocking wall structures 23 and the plurality of second blocking wall structures 33 have the same height to facilitate disposing of the transparent cover 50. Compared with conventional non-invasive blood glucose monitoring modules that require additional cutting operations to form grooves on the packaging structure and the transparent cover for setting up the blocking wall structures, the non-invasive blood glucose monitoring module with high optical transmittance 1 of the present invention can maintain an integrity of the packaging structure 40 and the transparent cover 50, prevent damages to the substrate 10 and related components, and improve the optical transmittance of the overall monitoring module. In addition, the non-invasive blood glucose monitoring module with high optical transmittance 1 of the present invention not only has a blocking wall structure between any light source set and its corresponding photosensitive element, but also has a blocking wall structure between those two light-emitting elements of any light source set, so as to avoid light interference and to improve the accuracy of the blood glucose detection.

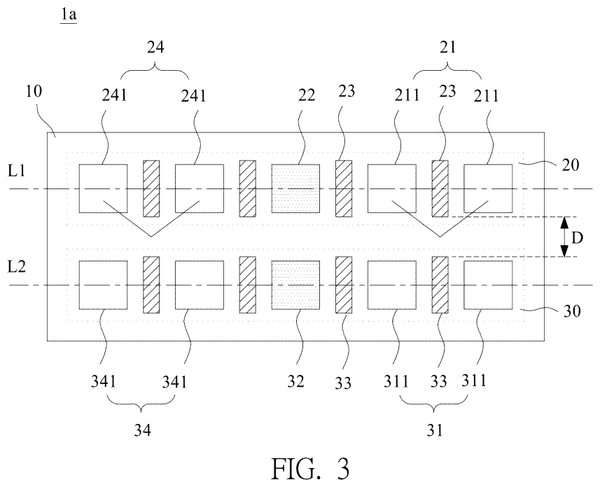

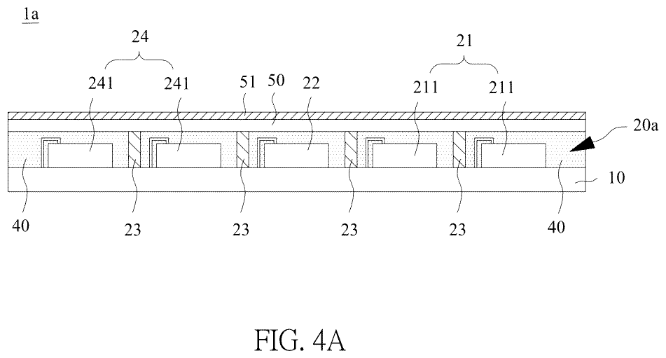

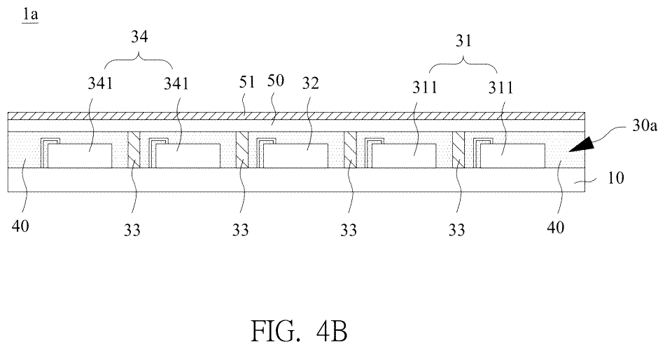

Please refer to FIG. 3, FIG. 4A and FIG. 4B together. FIG. 3 is a top view of a second embodiment of the non-invasive blood glucose monitoring module with high optical transmittance according to the present invention. FIG. 4A is a cross-sectional view along the first straight line L1 shown in FIG. 3 of the second embodiment of the non-invasive blood glucose monitoring module with high optical transmittance according to the present invention. FIG. 4B is a cross-sectional view along the second straight line L2 shown in FIG. 3 of the second embodiment of the non-invasive blood glucose monitoring module with high optical transmittance according to the present invention. This embodiment is a modification of the aforementioned first embodiment, with mainly adding other light source sets in the first detecting unit and in the second detecting unit. As shown in FIG. 3, FIG. 4A and FIG. 4B, in this embodiment, the first detecting unit 20a of the non-invasive blood glucose monitoring module with high optical transmittance 1a of the present invention further includes a third light source set 24, and the second detecting unit 30a further includes a fourth light source set 34.

The first light source set 21 and the third light source set 24 of the first detecting unit 20a are arranged symmetrically relative to the first photosensitive element 22. That is to say, the first light source set 21 and the third light source set 24 are respectively arranged on both sides of the first photosensitive element 22. The third light source set 24 has two third light-emitting elements 241. A default distance is set between the two third light-emitting elements 241, and each third light-emitting element 241 emits light with the first wavelength or the second wavelength. The first photosensitive element 22 is used to sense the light emitted by the first light source set 21 and the third light source set 24 correspondingly. Each third light-emitting element 241 can also be electrically connected to the substrate 10 through a bonding wire, and each third light-emitting element 241 can also be packaged within the packaging structure 40. A minimum distance from each third light-emitting element 241 to the first photosensitive element 22 is different. In this embodiment, the first photosensitive element 22, the first light-emitting elements 211 and the third light-emitting elements 241 of the first detecting unit 20a are arranged along the first straight line L1.

In response to the arrangement of the third light source set 24, more first blocking wall structures 23 can be added between the two third light-emitting elements 241 of the third light source set 24 and between the third light source set 24 and the first photosensitive element 22, to provide the corresponding light-blocking effect.

The second light source set 31 and the fourth light source set 34 of the second detecting unit 30a are symmetrically arranged relative to the second photosensitive element 32. That is to say, the second light source set 31 and the fourth light source set 34 are respectively arranged on both sides of the second photosensitive element 32. The fourth light source set 34 has two fourth light-emitting elements 341. A default distance is set between the two fourth light-emitting elements 341, and each fourth light-emitting element 341 emits light with the first wavelength or the second wavelength, and the wavelength of the light emitted by each fourth light-emitting element 341 is different from the wavelength of the light emitted by each third light-emitting element 241. For example, if each third light-emitting element 241 emits light with the first wavelength, then each fourth light-emitting element 341 emits light with the second wavelength. Based on different designs, the value of the first wavelength and the value second wavelength can be exchanged with each other. The second photosensitive element 32 is used to sense the light emitted by the second light source set 31 and the fourth light source set 34 correspondingly.

The fourth light-emitting elements 341 can also be electrically connected to the substrate 10 through bonding wires, and each fourth light-emitting element 341 is also packaged within the packaging structure 40. A minimum distance from each fourth light-emitting element 341 to the second photosensitive element 32 is different. In this embodiment, the second photosensitive element 32, the second light-emitting elements 311 and the fourth light-emitting elements 341 of the second detecting unit 30a are arranged along the second straight line L2, and the second straight line L2 is parallel to the first straight line L1. Accordingly, the first detecting unit 20a and the second detecting unit 30a are arranged side by side with each other. That is to say, the two first light-emitting elements 211 of the first light source set 21, the two third light-emitting elements 241 of the third light source set 24 and the first photosensitive element 22 of the first detecting unit 20a, and the two second light-emitting elements 311 of the second light source set 31, the two fourth light-emitting elements 341 of the fourth light source set 34 and the second photosensitive element 32 of the second detecting unit 30a can form an arrangement structure similar to a 2×5 matrix. The first detecting unit 20a and the second detecting unit 30a each forms an x-direction row, while the first photosensitive element 22 and the second photosensitive element 32, each first light-emitting element 211 and the adjacent second light-emitting element 311, and each third light-emitting element 241 and the adjacent fourth light-emitting element 341 form y-direction rows respectively.

Similarly, in response to the arrangement of the fourth light source set 34, more second blocking wall structures 33 can be added between the two fourth light-emitting elements 341 of the fourth light source set 34 and between the fourth light source set 34 and the second photosensitive element 32, to provide the corresponding light-blocking effect.

Moreover, in other embodiments of the present invention, a plurality of the non-invasive blood glucose monitoring module with high optical transmittance 1 or 1a of the present invention can also be combined to form a larger blood glucose monitoring module. For example, if two of the aforementioned non-invasive blood glucose monitoring module with high optical transmittance 1 of the present invention are placed side by side, the detecting units can form an arrangement structure similar to a 4×3 matrix. If two of the aforementioned non-invasive blood glucose monitoring module with high optical transmittance 1a of the present invention are arranged side by side, and the detecting units can form an arrangement structure similar to a 4×5 matrix. By combinations of different numbers of the non-invasive blood glucose monitoring module with high optical transmittance 1 or 1a of the present invention, the detection accuracy can be further improved, and even an effect of blood glucose detection through multiple wavelengths is provided.

The above implementations are only auxiliary descriptions, and are not intended to limit the embodiments of the application subject or the applications or uses of the embodiments. In addition, although at least one illustrative example has been presented above, it should be understood that the present invention can still have a large number of variations. It should also be understood that the embodiments described herein are not intended to limit the scope, use, or configuration of the requested subject matter in any way. On the contrary, the foregoing embodiments will provide a convenient guide for those skilled in the art to implement one or more embodiments. Furthermore, various changes can be made to the function and arrangement of the components without departing from the scope defined by the patent claims, and the scope of the patent claims includes known equivalents and all foreseeable equivalents at the time that the patent application is filed.

What is claimed is:

1. A non-invasive blood glucose monitoring module with high optical transmittance, comprising:
a substrate,
a first detecting unit, disposed on the substrate, including
a first light source set, having two first light-emitting elements and each first light-emitting element emitting light with a first wavelength,
a first photosensitive element, correspondingly receiving the light emitted from the first light source set, and each of the first light-emitting elements having a different minimum distance relative to the first photosensitive element, and
a plurality of first blocking wall structures, located between the two first light-emitting elements and between the first light source set and the first photosensitive element, and
a second detecting unit, disposed on the substrate, including
a second light source set, having two second light-emitting elements, and each second light-emitting element emitting light with a second wavelength, wherein the second wavelength is different from the first wavelength,
a second photosensitive element, correspondingly receiving the light emitted from the second light source set, and each of the second light-emitting elements having a different minimum distance relative to the second photosensitive element, and
a plurality of second blocking wall structures, located between the two second light-emitting elements and between the second light source set and the second photosensitive element.

2. The non-invasive blood glucose monitoring module with high optical transmittance as claimed in claim 1, wherein a length of each of the first blocking wall structures is longer than a length of each of the first light-emitting elements and longer than a length of the first photosensitive element, and wherein a length of each of the second blocking wall structures is longer than a length of each of the second light-emitting elements and longer than a length of the second photosensitive element.

3. The non-invasive blood glucose monitoring module with high optical transmittance as claimed in claim 1, wherein any first blocking wall structure of the first detecting unit is not connected to the correspondingly closest one of the second blocking wall structures of the second detecting unit, thus to maintain a gap thereof.

4. The non-invasive blood glucose monitoring module with high optical transmittance as claimed in claim 1, wherein the first photosensitive element and each of the first light-emitting elements of the first detecting unit are arranged along a first straight line, the second photosensitive element and each of the second light-emitting elements of the second detecting unit are arranged along a second straight line, and the first straight line is parallel to the second straight line.

5. The non-invasive blood glucose monitoring module with high optical transmittance as claimed in claim 4, wherein the first detecting unit further includes a third light source set, the first light source set and the third light source set are arranged symmetrically relative to the first photosensitive element, the third light source set has two third light-emitting elements, and each of the third light-emitting elements emits light with the first wavelength or the second wavelength, and wherein the second detecting unit further includes a fourth light source set, the second light source set and the fourth light source set are arranged symmetrically relative to the second photosensitive element, the fourth light source set has two fourth light-emitting elements, each of the fourth light-emitting elements emits light with the first wavelength or the second wavelength, and the wavelength of the light emitted from each of the fourth light-emitting elements is different from the wavelength of the light emitted from each of the third light-emitting elements.

6. The non-invasive blood glucose monitoring module with high optical transmittance as claimed in claim 1, wherein a viscosity of the material of the first blocking wall structures and the second blocking wall structures is greater than 10000 cps.

7. The non-invasive blood glucose monitoring module with high optical transmittance as claimed in claim 1, further comprises a packaging structure to package each of the first light-emitting elements, the first photosensitive element, each of the second light-emitting elements and the second light-emitting element, wherein a height of the package structure is larger than heights of each of the first light-emitting elements, of the first photosensitive element, of each of the second light-emitting elements and of the second light-emitting element.

8. The non-invasive blood glucose monitoring module with high optical transmittance as claimed in claim 7, wherein the packaging structure, each of the first blocking wall structures and each of the second blocking wall structures have the same height.

9. The non-invasive blood glucose monitoring module with high optical transmittance as claimed in claim 7, wherein a packaging material of the packaging structure has a viscosity between 1000 cps and 3000 cps and has an optical transmittance greater than 95%.

10. The non-invasive blood glucose monitoring module with high optical transmittance as claimed in claim 1, wherein the first wavelength is between 1000 nm and 1400 nm and the second wavelength is between 1400 nm and 1800 nm.

11. The non-invasive blood glucose monitoring module with high optical transmittance as claimed in claim 1, further comprising a transparent cover disposed on the first detecting unit and the second detecting unit.
